(19) United States
(12) Patent Application Publication
(10) Pub. No.: US 2006/0147527 A1
(43) Pub. Date: Jul. 6, 2006

(54) Controlled release preparations comprising tramadol and topiramate

(75) Inventors: Dieter Bachmann, Neuhausen (CH); Reza Eivaskhani, Freiburg i. br (DE); Christian Braun, Rielasingen (DE); Rene Spycher, Benken (CH); Brian Strong, Merishausen (CH)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: CILAG AG, Schaffhausen (CH)

(21) Appl. No.: 10/538,946

(22) PCT Filed: Dec. 12, 2003
(86) PCT No.: PCT/EP03/14474

§ 371 Date: Dec. 27, 2005

(30) Foreign Application Priority Data
Dec. 13, 2002 (EP) 02080325.0
Jan. 10, 2003 (EP) 03075123.4

(51) Int. Cl. A61K 31/7024; A61K 31/137; A61K 9/22

(52) U.S. Cl. 424/468; 514/23; 514/649

(57) ABSTRACT

This invention relates to an oral pharmaceutical preparation, suitable for dosing every 24 hours, comprising a substrate, which substrate comprises a pharmaceutically effective amount of tramadol or a salt thereof and a pharmaceutically effective amount of topiramate and wherein said substrate may be coated with a controlled release coating; said preparation having a specific dissolution rate in vitro.

### FIELD OF THE INVENTION

This invention relates to an oral pharmaceutical preparation, suitable for dosing every 24 hours, comprising a substrate, which substrate comprises a pharmaceutically effective amount of tramadol or a salt thereof and a pharmaceutically effective amount of topiramate and wherein said substrate may be coated with a controlled release coating; said preparation having a specific dissolution rate in vitro.

### BACKGROUND OF THE INVENTION

A number of effective anticonvulsants including the compound 2,3:4,5-bis-O-(1-methylethylidene)-β-D-fructopyranose sulfamate, also known as topiramate, have been disclosed in U.S. Pat. No. 4,513,006. Topiramate is useful in the treatment of human epilepsy in that it is effective as adjunctive therapy or as monotherapy in treating simple and complex partial seizures and secondarily generalized seizures. Topiramate is currently marketed for the treatment of simple and complex partial seizure epilepsy with or without secondary generalized seizures.

Recent preclinical studies on topiramate have revealed previously unrecognised pharmacological properties, which suggest that topiramate is effective in treating some other disorders. One of these is neuropathic pain, which remains one of the “frontiers” of pain management. There is a significant unmet need for efficacious and tolerable pharmacotherapy, making neuropathic pain an area of intense research interest. The term “neuropathic pain” is applied to any acute or chronic pain syndrome in which the sustaining mechanism for the pain is believed to involve abnormal transmission (peripheral) or processing (central) of somatosensory input.

U.S. Pat. No. 5,760,007 discloses topiramate as useful for the treatment of neuropathic pain. U.S. Pat. No, 5,935,933 discloses anticonvulsant derivatives useful in treating neuropathic pain, including but not limited to neuralgia.

A class of analgesic cycloalkanol-substituted phenol esters having a basic amine group in the cycloalkylring, are disclosed in U.S. Pat. No. 3,652,589. Among these is the compound (1R,2Ror 1S,2S)-2-[(dimethylamino)methyl]-1-(3-methoxyphenyl) cyclohexanol, commonly known as tramadol, which is specifically disclosed therein.

A series of articles pertaining to the pharmacology, toxicology and clinical studies of tramadol are found in Arzneim. Forsch., (Drug Res.), 1978, 28(1), 114. The Abstracts of the VIIth World Congress on Pain, Apr. 1-6 (1990), disclose that tramadol hydrochloride is an orally active pure agonist opioid analgesic. However, clinical experience indicates that tramadol lacks many of the typical side effects of opioid agonists, e.g., respiratory depression, constipation, tolerance and abuse liability. Tramadol's opioid activity without the typical side effects thereof makes it a very unique drug. Tramadol is currently marketed as an analgesic.

As an analgesic, tramadol has been combined with both opioid and non-opioid analgesic drugs. Such compositions have exhibited synergistic effects in treating pain while using less of each ingredient to produce an equivalent degree of analgesia. Specifically, U.S. Pat. No. 5,516,803 discloses the composition of tramadol and a NSAID, particularly ibuprofen. U.S. Pat. No. 5,468,744 discloses tramadol plus any of oxycodone, codeine or hydrocodone and U.S. Pat. No. 5,336,691 discloses tramadol in combination with acetaminophen.

WO-01/13904 relates to a pharmaceutical composition comprising a combination of a tramadol material and an anticonvulsant drug, in particular topiramat, and to the pharmacological use of the composition in treating conditions of pain and neurologic or psychiatric disorders. The composition has improved properties, requiring less of each ingredient and producing a synergistic effect. EP-A-699 436 discloses controlled realease preparations for oral administration containing tramadol or a salt thereof with a specific in vitro dissolution profile. EP-A-914 823 discloses sustained release preparations with tramadol hydrochoride as active ingredient with a fatty alcohol as matrix forming agent. U.S. Pat. No. 5,427,799 concerns sustained release compositions comprising an active ingredient, which may be an analgesic, in mixture with xanthan gum.

In animal studies using the Chung model of post-nerve constriction injury, both tramadol and topiramate are significantly active and reach 100% MPE as the dose is escalated. When topiramate and tramadol are co-administered in this model the ED₅₀ of both drugs is dramatically reduced, suggesting synergy of analgesic effect. The degree of synergy varies across ratios in this model with those ratios in which tramadol predominates displaying the greatest synergy.

In order to be effective in the treatment of neuropathic pain, it is necessary that sufficient amounts of both tramadol and topiramate are present at the receptor sites, in particular in the concentrations and concentration ratios required to produce the synergistic effect described in WO-01/13904. A particularly effective w/w ratio of both ingredients is in the range of about 1:1.5 to about 1:5, in particular from about 1:3 to about 1:5 of topiramate:tramadol. This w/w ratio should be reflected in the blood plasma levels of both these ingredients. Maintaining said blood plasma levels within this concentration ratio is a desirable goal to achieve. This goal becomes even more challenging when sustained release formulations are desired for administration every 12 or 24 hours, this because of the different half-life values of both agents. Indeed, the metabolisation rate of topiramate is a relatively slow while that of tramadol is relatively fast. Thus, without special measures, the blood plasma level of topiramate will be largely in excess to that of tramadol so that the latter no longer is present in a sufficient amount to be effective and the synergistic effect no longer is present.

Compositions including combinations of topiramate and tramadol have been found to be relatively unstable, in particular due to the instability of topiramate. There is therefore a need to provide formulations containing both topiramate and tramadol, with increased stability. Moreover there is a need to provide a composition that releases both topiramate and tramadol in a manner such that the blood plasma levels are sufficient for both active ingredients to be effective and to act synergistically. Providing such compositions is an object of this invention. A further object of this invention is to provide a method for treating conditions of pain, in particular neuropathic pain in mammals.

### SUMMARY OF THE INVENTION

This invention relates to a controlled release oral pharmaceutical preparation suitable for dosing every 24 hours comprising a substrate comprising a pharmaceutically effective amount of tramadol or a salt thereof and a pharmaceutically effective amount of topiramate; said preparation having a dissolution rate in vitro when measured using the Ph. Eur. Paddle Method at 75 rpm in 900 ml 0.05 M phosphate buffer at 37° C. and using high performance liquid chromatography (HPLC):
between 15 and 35% tramadol released after 1 hour;
between 28 and 48% tramadol released after 2 hours;
between 47 and 67% tramadol released after 4 hours;
between 68 and 88% tramadol released after 8 hours;
between 79 and 99% tramadol released after 12 hours;
between 86 and 105% tramadol released after 18 hours;
about 100% tramadol released after 24 hours; by weight,
said preparation providing a therapeutic effect for about 24 hours after oral administration.

In a preferred aspect there is provided an oral pharmaceutical preparation as defined above, said preparation having a dissolution rate as follows:
between 20 and 30% tramadol released after 1 hour;
between 33 and 43% tramadol released after 2 hours;
between 52 and 62% tramadol released after 4 hours;
between 73 and 83% tramadol released after 8 hours;
between 84 and 94% tramadol released after 12 hours;
between 91 and 100% tramadol released after 18 hours;
about 100% tramadol released after 24 hours; by weight.

In a most preferred aspect there is provided an oral pharmaceutical preparation as defined above, said preparation having a dissolution rate as follows:
about 25% tramadol released after 1 hour;
about 38% tramadol released after 2 hours;
about 57% tramadol released after 4 hours;
about 78% tramadol released after 8 hours;
about 89% tramadol released after 12 hours;
about 96% tramadol released after 18 hours;
about 100% tramadol released after 24 hours; by weight.

In preferred embodiments the tramadol salt is tramadol hydrochloride.

In further embodiments the said substrate is a suitable matrix material in which tramadol or its salt form is incorporated, said matrix material preferably comprising xanthan gum.

In a further aspect, the invention concerns pharmaceutical preparations as described herein comprising two or more phases. In certain embodiments, the major part of the tramadol or its salt form and of the topiramate are in different phases of the said pharmaceutical preparations. In these embodiments at least one phase may contain either the major part of topiramate or the major part of tramadol or a salt-form thereof. In particular embodiments, one phase contains the major part of tramadol or a salt thereof and another phase contains the major part of topiramate. Further particular embodiments are pharmaceutical preparations that take the form of a bi-phasic tablet having a phase that comprises the major part of topiramate and another phase that comprises the major part of tramadol or a salt form thereof. The phases in these embodiments may take the form of layers.

In a specific aspect the invention concerns pharmaceutical preparations as described herein comprising two or more phases, wherein the tramadol or its salt form and the topiramate are in different phases of the said pharmaceutical preparations and none of the phases contains as well topiramate as tramadol or a salt-form thereof. In particular embodiments, one phase contains the tramadol or a salt thereof and another phase contains the topiramate. Further particular embodiments are pharmaceutical preparations that take the form of a bi-phasic tablet having a phase that comprises the topiramate active ingredient and another phase that comprises the tramadol active ingredient or a salt form thereof.

In a particular aspect this invention concerns pharmaceutical preparations as defined herein, wherein said preparations are bi- or multi-layer tablets and wherein none of the layers contains as well topiramate as tramadol or a salt-form thereof. In particular embodiments, the previously mentioned pharmaceutical preparations take the form of a bilayer tablet having a phase that comprises the topiramate active ingredient and another phase that comprises the tramadol active ingredient or a salt form thereof.

In a further aspect, there is provided a bi- or multiphasic tablet according to the invention containing an effective amount of topiramate having at least one phase or layer that contains from about 20% to about 100%, in particular from about 30% to about 90% or from about 50% to 80% of polymeric matrix material. The latter preferably is xanthan gum.

In a particular embodiment, the tablets according to the present invention are coated with an appropriate coating. The coating may be for taste masking or for other purposes.

The invention also provides preparations as defined herein which are capsules or sachets. The topiramate and/or the tramadol containing phase or phases in these embodiments may take the form of pellets.

In another aspect the invention concerns a process for manufacturing the oral pharmaceutical preparation described herein comprising mixing tramadol hydrochloride, being incorporated in a suitable controlled release substrate, and topiramate, preferably formulated in a suitable carrier material solid form.

In a further aspect there is provided a process for manufacturing a bi- or multiphasic tablet in accordance with the invention, comprising compressing two or more pre-shaped phases in an appropriate compressing apparatus.

In a further aspect there is provided a process for manufacturing a bi- or multilayer tablet in accordance with the invention comprising compressing a suitable topiramate containing composition as to form a layer, laying tramadol containing matrix material on this topiramate containing layer, compressing the whole, and if desired laying further compositions of topiramate and/or further tramadol containing matrix material thereon and each time subjecting the whole to a compression and if further desired coating the thus prepared dosage form.

In a further aspect there is provided a process for manufacturing a bi- or multilayer tablet in accordance with the invention comprising compressing tramadol containing matrix material as to form a layer, laying a suitable topiramate containing mixture on this tramadol containing matrix material layer, compressing the whole, and if desired laying further compositions of topiramate and/or further tramadol matrix material thereon and each time subjecting the whole to a compression and if further desired coating the thus prepared dosage form.

Furthermore, the invention concerns a method of treating a warm blooded animal suffering from neuropathic pain, said method comprising the administration of a pharmaceutical preparation as described herein.

### DETAILED DESCRIPTION OF THE INVENTION

As mentioned herein, any % is weight-by-weight, relative to the total weight of the preparation or the formulation.

Tramadol is the compound (1R,2R or 1S,2S)-2-[(dimethylamino)methyl]-1-(3-methoxyphenyl)-cyclohexanol. Preferably tramadol is used as a salt form, in particular its hydrochloride salt. Tramadol is commercially available from Gruenenthal or may be made by the process described in U.S. Pat. No. 3,652,589, which is herein incorporated by reference.

Topiramate is the compound 2,3,4,5-bis-O-(1-methylethylidene)-β-D-fructopyranose sulfamate and can be prepared according to processes described in U.S. Pat. No. 4,513,006.

The controlled release preparation should release tramadol in vitro in the quantities outlined above. These are obtained by measurement using the Ph. Eur. Paddle Method at 75 rpm in 900 ml 0.05 M phosphate buffer with a pH value of 6.8 (USP) at 37° C. and using high performance liquid chromatography (HPLC). In the latter a suitable detection system is used such as, for example, UV detection at an appropriate wavelength, e.g. at 270 nm in the case of tramadol, or with a refractive index detector. Alternatively, release of the active ingredients and in particular release of tramadol can be measured by using in situ measurement with a fiber optic dissolution system, using the second derivative correction method at a suitable wavelength range, which, in case of tramadol is in the range of 283 to 289 nm.

The controlled release preparations according to the invention should be suitable for dosing every 24 hours. The ‘term suitable for dosing every 24 hours’ means that the dosage forms should be such that they can be administered every 24 hours and give effective blood plasma concentrations of both active ingredients such that they are effective to treat neuropathic pain over a period of 24 hours. The controlled release preparations according to the invention can be dosed every 24 hours but also can be dosed differently, e.g. every 12 hours (b.i.d.) or every 8 hours (t.i.d.).

As mentioned above, in order to act synergistically, the ratios of the blood plasma levels of topiramate:tramadol should be within certain ranges and in particular the blood plasma levels of these ingredients should be in the range of about 1:1.5 to about 1:5, in particular from about 1:3 to about 1:5 of topiramate:tramadol. It has been found that an optimum ratio for these ingredients is about 1:3 topiramate:tramadol. It further has been found that when tramadol is released in vitro in the quantities outlined above, upon multiple administrations during specific periods of time, e.g. every 12 hours, or, which is preferred, every 24 hours, the plasma concentrations of tramadol in vivo reach a steady state and are constant within certain ranges during an extended period of time. It has additionally been found that in pharmaceutical preparations as described herein, containing tramadol or a salt thereof and topiramate, when the release of tramadol follows the release pattern as outlined above, the ratio of the plasma concentrations of topiramate vis a vis tramadol is constant within certain ranges. This ratio has been found to approximate 1:3 (w/w) or upon selection of the appropriate concentrations of the topiramate and tramadol, or its salt form, and of the substrate and other carriers in the pharmaceutical dosage form, this ratio may be 1:3 or about 1:3. This equally means that upon multiple administrations of the pharmaceutical preparations of this invention, also during specific periods of time, e.g. every 12 hours, or, which is preferred, every 24 hours, the plasma concentrations of topiramate in vivo reach a steady state and are constant within certain ranges during a long period of time. This will allow both agents to act synergistically and therefore to be effective in combating neuropathic pain during a longer period of e.g. 12 or even 24 hours upon administration of the pharmaceutical preparations in accordance with the invention.

As used herein, ‘constant within certain ranges’ means that there can be small fluctuations of the plasma concentrations or of the ratio of the plasma concentrations within an acceptable range, e.g. within 30% in particular, within 20%, fuirther in particular within 10%. This alternatively can be expressed by the ‘fluctuation index’ which is defined as follows:

$${F}_{i} = \frac{{C}_{max} - {C}_{min}}{{C}_{Av}}$$

wherein Fᵢ is the fluctuation index, Cₘₐₓ the maximal plasma concentration, Cₘᵢₙ the minimal plasma concentration, and C_{AV} the average plasma concentration. The fluctuation index can vary but for example is 1.3, or 1.2 or even 1.1.

The in vitro release quantities of tramadol outlined above are based on an analysis of the in vivo plasma concentration data of topiramate and on the in vitro/in vivo correlation of plasma concentrations and in vitro release quantities of tramadol. Administration of an effective amount of topiramate will show a particular course of the plasma concentrations. Ideally, tramadol should follow the same course so that the ratio of the plasma concentrations of both agents remains more or less constant. By measuring the correlation of plasma concentrations and in vitro release quantities of tramadol it has been found that it is possible to predict which plasma concentration correlates with a given in vitro release quantity and vice versa (this method can be referred to as reversed IVIVC, i.e. reversed in vitro in vivo correlation). This allows a reverse calculation of the in vitro release quantities that would cause in vivo plasma concentrations, which run in parallel with the plasma concentrations of topiramate.

This invention relates to controlled release oral pharmaceutical preparations as specified herein comprising a substrate comprising a pharmaceutically effective amount of tramadol or a salt thereof and a pharmaceutically effective amount of topiramate. As used herein, the term ‘substrate’ refers to any material or combination of materials, or forms thereof, that results in the specified release pattern of tramadol.

Typically, the pharmaceutical preparations according to the present invention comprise tramadol or its salt form in a suitable controlled release form, which may be any form that affords release of tramadol within the ranges specified above.

In certain embodiments, the preparations of the invention comprise controlled release forms wherein the tramadol or its salt form is incorporated in a suitable matrix, which may be a controlled release matrix or a normal release matrix having a controlled release coating. The controlled release form may take various forms, e.g. particles of different sizes, pellets (or beads), tablets, phases within a larger unit such as layers or sections of other shape within a larger unit (e.g. as in a multi-layer or a bull-eye tablet). A number of such formats as well as the unit dosage forms in which these can be incorporated will be outlined in more detail hereinafter.

As used herein the term ‘phase’ refers to a defined three dimensionally shaped section in a tablet dosage form that contains the same material and wherein each phase is separated from the other. Examples of phases are layers, which are incorporated in bi- or multi-layer tablets. Other examples are cylindrical, spherical or other tridimensionally shaped sections that can be present in tablets. This gives rise to different tablet formats such as the so-called ‘bull-eye’ tablets, or concentric tablets (a central cylindrically shaped section completely surrounded with one or more further cylindric layers (i.e. a ring-like combination), or ‘coated’ tablets wherein the coating is a layer completely surrounding a tablet nucleus and the like tablet formats. Preference is given to bi- or multi-layer tablets.

Preferred embodiments are tablets that contain at least two phases, in particular tablets that contain at least two layers.

In particular embodiments, the major part of the tramadol or its salt form and of the topiramate are in different phases of the said pharmaceutical preparations. In said embodiments a at least one phase may contain either the major part of topiramate or the major part of tramadol or a salt-form thereof. In particular embodiments, one phase contains the major part of tramadol or a salt thereof and another phase contains the major part of topiramate. As used herein, ‘major part’ means that the major quantity of the tramadol or its salt form or of topiramate is present in a particular phase. Preferably the term ‘major part’ refers to a situation where at least more than about 90% of the concerned active ingredient is present in a particular phase, for example more than 95%, or more than 98%, or more than 99%, or even more than 99.5%. The same applies to the situation take particular forms such as layers.

Most preferably a phase containing one of both active ingredients should contain only a minute amount of the other active ingredient, or even none of the other active ingredient, for example a phase may contain topiramate and a minute amount, e.g. less than 1%, or less than 0.5% of tramadol or a salt form thereof, or vice versa.

Preferably a phase comprising tramadol or a salt form thereof is adjacent to a phase containing topiramate.

Of particular interest are tablets that are biphasic, the latter being preferred, or multiphasic, e.g. having 3, 4, 5 or more phases. At least one layer should comprise tramadol or a salt form thereof but in case of multiphasic tablets, more than one layer comprising tramadol or a salt form can be present. Of still further interest are those preparations in which one or more of the phases are layers.

Particularly preferred embodiments are tablets wherein topiramate is present in amounts from about 10 mg to 500 mg topiramate per unit, preferably from about 25 mg to about 200 mg of topiramate per unit, e.g. tablets having 25, 50, 100 or 200 mg per unit.

In a particular aspect, the tablets of the invention contain an effective amount of topiramate, wherein the tablets have at least one layer that contains from about 20% to about 100%, in particular from about 30% to about 90% or from about 50% to 80% of polymeric matrix material. The hygroscopic matrix material containing layer may contain other ingredients such as the ingredients mentioned hereinafter.

Controlled Release Matrix

Where the matrix is a controlled release it may comprise suitable digestible hydrophilic or hydrophobic polymeric or non-polymeric materials.

Examples of such polymeric materials are hydrophilic or hydrophobic polymers, such as polysaccharides, in particular gums (further in particular pH dependent gums), cellulose ethers, especially alkylcelluloses, in particular C1-C₆ alkyl cellulose, especially ethyl cellulose, acrylic resins, protein-derived materials, polyalkylene glycols, and the like. Preferred are the polysaccharide gums such as xanthan gum.

Examples of non-polymeric materials that can be used are digestible lipids having a long chain alkyl moiety, which may be straight or branched, saturated or unsaturated, substituted or unsubstituted. Of particular interest are C₈₋₅₀, especially C₁₂₋₄₀ lipids. Examples comprise fatty acids, fatty alcohols, glyceryl esters of fatty acids, mineral and vegetable oils and waxes. Lipids having a melting point of between 25 and 90° C. are preferred.

The tramadol controlled release form may conveniently contain between 1% and 90%, in particular from 10% to 90%, further in particular from 20% to 80% (by weight) of one or more hydrophilic or hydrophobic polymers or digestible lipids. In the embodiments where the polymeric material is a polysaccharide gum such as xanthan gum, alginate or gum Arabic, the preparation may contain between 20% and 90%, in particular from 30% to 80%, (by weight) of xanthan gum. As used herein, ‘alginate’ refers to alginate or its salts, in particular to its alkali metal salts such as sodium or potassium salts. In the embodiments containing polyalkylene glycols, the preparation may in particular contain up to 60% (by weight) of one or more polyalkylene glycols. In further particular embodiments, the preparation may contain up to 60% (by weight) of at least one digestible, long chain lipid.

Of particular interest are controlled release matrixes comprising xanthan gum optionally in mixture with other gums, in particular with other pH dependent gums such as, for example, alginate.

Optionally, the controlled release matrix may also contain other pharmaceutically acceptable ingredients which are conventional in the pharmaceutical art such as diluents (in particular lactose), lubricants, binders, granulating aids, colorants, flavorants, surfactants, pH adjusters, anti-adherents and glidants (e.g. colloidal silica), and plasticizers (e.g. dibutyl sebacate) and other suitable ingredients (e.g. ammonium hydroxide, oleic acid).

The tramadol controlled release form may conveniently be film coated using any film coating material conventional in the pharmaceutical art. A film coat is added e.g. as a finish, for coloring purposes or taste masking or a combination of these. Preferably an aqueous film coating is used.

Alternatively, the tramadol controlled release form may comprise a normal release matrix, as a core, and further having a controlled release coating. In such embodiments the tramadol form may be prepared via art known procedures, e.g. by a suitable granulation process followed by compression, or by direct compression, followed by a coating step with a coating material that ensures controlled release. In such preparations, the tramadol normal release section or core of the preparation may contain any of the usual ingredients usually employed to make such normal release sections or cores. Any of the ingredients mentioned hereinafter with respect to the incorporation of topiramate in the preparations of the invention can be conveniently employed.

The release profile of tramadol can be adjusted in a number of ways. For instance a higher loading of the drug will be associated with increased initial release rates. By selecting particular ingredients and by controlling the relative amounts thereof in the preparation it is possible to adjust the release profile of the tramadol. Such particular ingredients for example are the matrix materials mentioned above, e.g. the polymeric materials mentioned above.

Pellets

In further embodiments, the tramadol controlled release form comprises spherical pellets containing the active ingredient and a spheronizing agent. The pellets may be film-coated or not. The spheronizing agent may be any suitable pharmaceutically acceptable material, which can be spheronized together with the active ingredient to form pellets. The term ‘spherical pellet’ is meant to comprise pellets, beads or spheroids that are more or less of regular shape. In particular embodiments of the invention the shape is round or about round, i.e. having or approaching the shape of a small sphere.

The average size of the pellets may vary but preferably the diameter is in the range of about 0.1 mm to 3 mm, in particular from about 0.5 mm to about 2 mm, more preferably about 1 mm.

The size distribution of the pellets may vary but in general it is preferred that it has limited variation. It may vary between within a range of 10 to 20%. The size distribution may vary in a statistical manner, i.e. in a bell-shaped curve wherein e.g. 90% or e.g. 95% of the number of pellets are within a size range that varies between about 10% to about 20% of the average sizes mentioned above.

The tramadol or its salt form is present in an amount, which is in the range of from about 0.1 to about 50%, in particular from about 1 to about 40%, more in particular from about 10 to about 35%, w/w relative to the total weight of the pellet.

The pellets of the invention may further comprise an appropriate carrier which may be any carrier known in the art used for making pellets. Particular carrier materials are spheronizing agents that may be any suitable pharmaceutically acceptable material, which may be spheronized together with the active ingredient to form pellets. A preferred spheronizing agent is microcrystalline cellulose. The microcrystalline cellulose used may suitably be, for example, the product sold under the tradename ‘Avicel™’. The spheronizing agent is present in an amount, which is in the range of from about 25% to about 90%, in particular from about 35% to about 70% w/w, relative to the total weight of the pellet.

Optionally the pellets may contain other pharmaceutically acceptable ingredients such as binders, bulking agents and colorants. Suitable binders, some of which may also contribute to the controlled release properties of the pellets, include water-soluble polymers, e.g. water-soluble hydroxyalkyl celluloses such as hydroxypropyl cellulose, or water insoluble polymers, such as acrylic polymers or copolymers, or alkyl celluloses such as, for example, ethylcellulose. Suitable bulking agents include lactose or colloidal silicon dioxide. The amount of these other ingredients in the pellets will be relatively small, e.g. lower than 30%, or 20%, or even lower than 10% or 5% w/w relative to the total weight of the pellet.

The pellets for use in the preparations of the present invention are made by an extrusion process followed by spheronization. The mixture used in the extrusion process comprises active ingredient, a suitable carrier material and other optional ingredients, and a suitable lubricant. The lubricant usually is water and the mixture for extrusion typically is converted into a granulate. After extrusion, the extrudate is spheronized to obtain pellets. If desired, the latter may be coated with a suitable coating material.

Tramadol is known to usually form a sticky mass upon contact with water and/or the other excipients used in the extrudate mixture. More in particular, tramadol seems to act as an additional binder in the mixture that is extruded and spheronized. This can be avoided by the addition of a dry lubricant. Apart from providing lubrication, the dry lubricant also allows the material to be extruded at a much lower moisture content thereby reducing the sticking observed in the spheronizer.

Further embodiments thus are spherical pellets for sustained release comprising tramadol or a salt thereof, a spheronizing agent and dry lubricant. In a further aspect, said pellets have a low water content. If desired, the pellets may be coated.

The dry lubricant in particular is a mono-, di- or triglyceride, or mixtures thereof. Suitable mono-, di- or triglycerides are the mono-, di- or triesters of glycerine and one or more fatty acids. The mono-, di- or triglycerides may contain the same or different fatty acid residues or mixtures thereof, e.g. technical mixtures obtained from saponification of natural oils. Of particular interest are fatty acid triglycerides wherein the fatty acid residue has from 12 to 30 carbon atoms and is saturated or partially unsaturated or may be substituted, e.g. with one or more hydroxy functions. Preferred are mono-, di- or triglycerides derived from C₁₈₋₃₀ fatty acids, in particular derived from C₂₂₋₂₆ fatty acids. Of particularly preferred interest are behenic acid mono-, di- or triglycerides.

The dry lubricant preferably is solid at room temperature and has a melting point or melting range which is in the range of 60 ° C. to 90° C., in particular is in the range of 70° C. to 80 ° C. A particularly suitable dry lubricant is the glyceride mixture sold under the trade name ‘Compritol™ 888ATO’ which is a mixture of glyceryl mono-, di- and tribehenate, the dibehenate fraction being predominant, and having a melting range of about 69-74° C.

Preferably, the dry lubricant is selected such that it does not impact the dissolution behavior of the active ingredient.

The dry lubricant is present in an amount, which is in the range of from about 2% to about 50%, in particular between 10% and 35% w/w, relative to the total weight of the pellet.

Of particular interest are pellets that have a low water content. In particular embodiments, the water contents in the pellets is lower than 5%, more in particular lower than 3%, w/w relative to the total weight of the pellet.

The tramadol spherical pellets, containing a dry lubricant, can be prepared by a process comprising extruding a mixture of the active ingredient with a suitable carrier in the presence of a dry lubricant and spheronizing the extrudate, wherein the dry lubricant is a triglyceride. The amount of dry lubricant in this mixture may vary but in general is comprised between 10% and 35% (w/w). A small amount of water may be added to the mixture. In a particular execution, the amount of water is 5% or lower, or 3% or lower, or 1.5% or lower, w/w, relative to the total weight of the mixture for extrusion. In a specific process the pellets are subsequently coated with a suitable coating.

The ingredients may be mixed together in any given sequence. In one embodiment, the dry lubricant is added to a mixture of active ingredient and the carrier material at room temperature. The mixture is subsequently extruded through a small orifice. The diameter of the latter is in relation to the size of the pellets that are eventually produced from the extrudate. In one embodiment, the diameter of the orifices is in the range of 0.5 mm to 2.0 mm. The extrusion may be done at slightly elevated temperature but preferably is performed without applied heating. The extrudated material is subsequently placed into a spheronizer where it is spun at high speed.

In specific embodiments of the invention, the pellets (or spheroids), with or without dry lubricant, are subsequently coated with a suitable coating using art known methods. The coating can either be a functional coating or a diffusion controlling coating.

A functional coating may be applied for e.g. taste masking, protection of the pellets, to have improved stability (shelf-life) or for identification (for example by coloring). Functional coating often will be film coating, using any film coating material conventional in the pharmaceutical art. Preferably an aqueous film coating is used.

Diffusion controlling coatings are designed to achieve a target release profile such as controlled or sustained release permitting release of the active ingredient at a controlled rate in an aqueous medium. Suitable controlled or sustained release coating materials include water-insoluble waxes and polymers such as polymethacrylates, for example the Eudragit™ polymers, or water insoluble celluloses, in particular alkyl celluloses such as ethylcellulose. Optionally, water-soluble polymers such as polyvinylpyrrolidone or water-soluble celluloses such as hydroxypropylmethyl-cellulose or hydroxypropylcellulose may be included. Further components that may be added are water-soluble agents such as polysorbate. Of particular interest is ethylcellulose (EC). Preferably, a suitable plasticizer is added. A coating material that is particularly suitable is the coating material sold under the trade name Surelease™ (Colorcon), which is a dispersion of ethylcellulose.

Because of the bitter taste of the tramadol active ingredient, the pellets may be coated for taste-masking purposes although this may be of less importance if the pellets are used in a capsule dosage form.

Tramadol Coated on Beads

Alternatively the tramadol or its salt-form may be coated onto inert non-pareil beads, in particular onto sugar beads, and the drug loaded beads coated with a material, which permits control of the release of the active ingredient into the aqueous medium.

Topiramate

The topiramate in the preparations can be present throughout the pharmaceutical preparations of the invention or in particular sections thereof. In particular embodiments it is present in one or more phases of the preparations. Preferably, the topiramate is present in one or more phases that do not contain tramadol.

The phases can take a variety of forms, e.g. sections in a tablet, or they can take the form of pellets. These forms can be prepared following art-known procedures. In the particular case of sections in a tablet preparation, procedures can be applied such as granulation followed by partial or complete compression, or direct partial or complete compression.

Usually, the topiramate is formulated into a suitable formulation. This is prepared by mixing topiramate with suitable ingredients into different formulation types such as powders, granulates, pellets and the like. The powder or granulate formulations may be compressed partially or completely to form appropriate phases for incorporation in bi- or multi-phasic preparations. Particular phases are layers for incorporation in bi- or multi-layer tablets. Most preferably, the topiramate formulation will be for immediate release, i.e. the ingredients and the formulation form are selected such that release of topiramate is as quickly and as complete as possible. Ideally, release is 100% after a short period of time, e.g. within ½ hour.

In tablet preparations, suitable tabletting excipients may be added e.g. one or more of the standard excipients such as diluents, lubricants, binding agents, flow aids, disintegrating agents, surface active agents or water soluble polymeric materials. Suitable diluents are e.g. microcrystalline cellulose, lactose and dicalcium phosphate. Suitable lubricants are e.g. magnesium stearate and sodium stearyl fumarate. Suitable binding agents are e.g. hydroxypropyl methyl cellulose, polyvidone and methyl cellulose. Suitable disintegrating agents are starch, sodium starch glycolate, crospovidone and croscarmalose sodium. Suitable surface active are Poloxamer 188®, Polysorbate 80 and sodium lauryl sulfate. Suitable flow aids are talc, colloidal anhydrous silica.

Double Layer Tablets

One particular execution of the controlled release preparations of the present invention are double layer (or bilayer) tablets. These comprise one layer containing tramadol dispersed in a suitable matrix and another layer that contains topiramate.

The topiramate containing layer preferably is composed of excipients typically used for topiramate oral dosage forms such as tablets. Examples of such excipients comprise any of those mentioned above in relation to the formulation of topiramate.

The tramadol layer comprises any of the controlled release matrix materials described above. The matrix may in particular comprise a polysaccharide, more in particular a gum, still more in particular xanthan gum. Or alternatively, the tramadol layer may consist essentially of polysaccharide, more in particular a gum, still more in particular xanthan gum.

The tramadol layer may contain from about 10 mg to 100 mg tramadol hydrochloride per unit, preferably from about 15 mg to about 75 mg of tramadol hydrochloride per unit, or from about 25 mg to about 65 mg of tramadol hydrochloride per unit. In case of application of tramadol-free base or other salts, an equivalent amount of active is used.

In particular embodiments of this invention, the tramadol layer contains an effective amount of tramadol, or a pharmaceutically acceptable salt thereof, dispersed in a matrix wherein the matrix contains from about 20% to about 90%, in particular from about 30% to about 80% of a polysaccharide, more in particular a gum, still more in particular xanthan gum. The percentages mentioned herein are w/w relative to the total weight of the dosage form.

The tramadol layer may additionally contain further ingredients such as the ingredients mentioned previously in relation to the topiramate layer, in particular starches, kaolin, lubricants, binders and the like. Preferred additional carriers are lubricants, e.g. magnesium stearate, flow enhancers or fillers, e.g. silica (silicon dioxide), fillers such as sugars, in particular lactose, titanium dioxide and the like.

In further particular embodiments of this invention, the tramadol layer contains as further ingredients lactose as a filler and magnesium stearate as a lubricant. Lactose is added to improve compressibility of the blend. Magnesium stearate is added to avoid tablet sticking on the lower or upper punch during the compression. The concentration of magnesium stearate in the tramadol layer may vary but good results are obtained when adding it in amounts ranging from about 0.5 to about 1.0% (w/w relative to the total weight of the dosage form). The concentration of lactose in the tramadol layer may vary but good results are obtained when adding it in amounts which range from about 5% to about 80%, preferably from about 10% to about 65%, more preferably from about 20% to about 50% (w/w relative to the total weight of the dosage form).

The tramadol layer can be prepared by mixing tramadol or its salt form with the polysaccharide, more in particular the gum, still more in particular with xanthan gum while adding optional ingredients. The latter may also be added after the mixing of tramadol and xanthan gum. The thus obtained mixtures are subsequently compressed, either by direct compression, which is preferred or by preparing a granulate and subsequent compression.

It has been found that when using tramadol and xanthan gum mixtures, the tablets can be prepared by direct compression. The mixtures for direct compression preferably contain a lubricant, in particular magnesium stearate. They may additionally contain a filler, in particular a sugar such as lactose. They may furthermore contain a flow enhancer such as colloidal silica (silicon dioxide). In the mixtures for direct compression the lubricant preferably is present in concentrations in the range of about 0.75% to about 1.0%. The filler is present in concentrations from about 5% to about 80%, preferably from about 10% to about 65%, more preferably from about 20% to about 50%. The flow enhancer is present in concentrations from about 0.4% to about 0.6%, preferably about 0.45% to about 0.50%. All percentages herein are w/w relative to the total weight of the tramadol containing phase or phases.

Particular embodiments of the invention are coated tablets, in particular film-coated tablets. Coated tablets are easier to swallow than uncoated tablet cores, are usually easier to distinguish from other tablets—in particular when the film-coat contains a dye or a pigment—, and may furthermore have an improved stability (shelf-life). In the present instance coating is mainly is for taste masking purposes because of the bitter taste of tramadol. Coatings are applied using art known methods using art known materials usually applied for this purpose. Particularly attractive coating products are based on suitable film-forming polymers such as hydroxypropylmethylcellulose (HPMC) or polyvinylalcohol (PVA). Preferably, a plasticizer is added. Examples of suitable plasticizers are polyethylene glycol or derivatives thereof such as polyethoxylated alkylglycerides, e.g. polyethoxylated stearyl monoglyceride, in particular the material sold under the trade name Macrogol™. Further ingredients may be added to the coating such as fillers, dyes or pigments, flavors, sweeteners and the like components. Examples of such further ingredients are lactose, titanium dioxide, starch and the like. Particularly suited as coating materials are the Opadry™ materials which mainly contain the before mentioned materials and further ingredients such as plasticizers, e.g. polyethylene glycol.

In a preferred embodiment first the tramadol layer is produced by direct compression whereupon topiramate granules are placed on top of the compressed tramadol layer as to form a second layer whereupon the whole is compressed to form a bi-layer tablet.

In particular embodiments there are provided bi-layer tablets comprising a tramadol layer and a topiramate layer wherein both layers are separated by a suitable layer that may function as an isolator. This third layer may be comprised of suitable inert materials such as cellulose or lactose. Such embodiments may be prepared by first producing the tramadol layer by partial or complete compression of a suitable tramadol containing mixture whereupon the isolator material is put on the tramadol layer followed by a second compression, whereafter a suitable topiramate containing mixture is put on top of the isolator layer as to form a third layer whereupon the whole is compressed to form a tri-layer tablet. The suitable tramadol containing mixture or suitable topiramate mixture may be a powder suitable for direct compression or a granulate obtained by a granulation process. The isolator layer may be desirable e.g. to avoid certain interactions between the components in each layer or to shield off humidity.

Multi-Layer Tablets

Further embodiments are multi-layer tablets having multiple layers of topiramate and tramadol, optionally separated by one or more isolator layers.

Further Tablet Formulations

In still further embodiments are tramadol tablets coated with a topiramate coating. In this type of preparations, a suitable core containing tramadol or a salt thereof in a controlled release form is coated with a topiramte-containing coating, e.g. by spraying with a suitable liquid formulation that contains topiramate. The core itself can be a tablet or another shaped phase.

Still further embodiments of the invention are so-called ‘bull-eye’ tablets, which are tablets with a cavity in which another tablet fits. The tablet with the cavity in particular is U-shaped. The tablet with a cavity may contain the tramadol and the other tablet the topiramate or vice versa. Bull-eye tablets can be made following art-known procedures using specially adapted punches in a tabletting machine.

In any of the preparations that are tablets, the latter may be coated with a suitable coating material.

Preparations with Pellets

Further embodiments of this invention are dosage forms comprising tramadol formulated in pellets, hereafter referred to as ‘tramadol pellets’. The tramadol pellets may be prepared according to methods as described above and may be coated, if desired.

The tramadol pellets in turn may be coated with a topiramate containing coating, e.g. by spraying the tramadol pellets with an appropriate formulation containing topiramate. These tramadol pellets with a topiramate coating may be filled into capsules.

The tramadol pellets can be filled in capsules together with an appropriate formulation of topiramate e.g. formulated as a powder, granulate, or formulated itself as a pellet. The tramadol pellets and the topiramate formulation may be filled into the capsule in any give sequence, first the tramadol pellets followed by the topiramate formulation or vice versa or the two together or the two together as a mixture, e.g. a mixture of topiramate and tramadol pellets.

In further embodiments there are provided capsules containing tramadol pellets and one or more topiramate tablets. The topiramate tablets will evidently be of such size and shape that it fits into a capsule. Preferably only one topiramate tablet is filled into one capsule.

In still further embodiments there is provided a so-called ‘capsule into capsule’ dosage form, i.e. a capsule containing a suitable topiramate formulation is put into a bigger capsule containing tramadol pellets. Or vice versa, a capsule containing tramadol pellets is put into a bigger capsule containing a suitable topiramate formulation. A suitable topiramate formulation can be a powder or a pellet formulation.

Still other embodiments are sachets filled with amounts of tramadol pellets and a suitable topiramate formulation.

In still another aspect, the invention concerns a process for manufacturing a pharmaceutical dosage from, said method comprising filling the tramadol pellets into a suitable container and further adding a suitable topiramate formulation. In a preferred aspect the container is a capsule. Another type of container is a sachet.

In a particular embodiment, the invention provides unitary dosage forms, which comprise tramadol hydrochloride pellets as described herein in an amount that is such that the dosage form contains an effective amount of tramadol hydrochloride. Particular embodiments of such dosage forms may contain from about 10 mg to 100 mg tramadol hydrochloride per unit, preferably from about 15 mg to about 75 mg of tramadol hydrochloride per unit, or from about 25 mg to about 65 mg of tramadol hydrochloride per unit.

The pharmaceutical preparations of the invention have a particular dissolution rate of the active ingredients in vitro and provide an effective therapeutic effect for a sufficiently long period of time, in particular such that the preparation is suitable for administering every 24 hours. Other dosing frequencies are also possible, e.g dosing every 12 hours.

In a further aspect, the invention concerns a method for treating conditions of pain, in particular neuropathic pain, or a method of treating neurological or psychiatric disorders, in mammals in need of such treatment, said method comprising the administration to sais mammals of a pharmaceutical preparation as described herein.

Preferably, the pharmaceutical preparations of the invention are suited for dosing every 24 hours.

### EXAMPLES

### Example 1

Double Layer Tablet Preparation:

Tramadol/Xanthan Gum Layer:

| Actives and Excipients | mg/Tablet |
|---|---|
| Tramadol | 45.00 |
| Xanthan gum | 160.00 |
| Lactose (Fast Flo ™) | 139.92 |
| Magnesium Stearate | 3.50 |
| Silicon Dioxide | 1.58 |
| Total | 350.00 |

Topiramate Layer:

| Active and Excipients | mg/Tablet |
|---|---|
| Topiramate | 15.00 |
| Lactose Monohydrate | 18.51 |
| Pregelatinized Starch | 3.84 |
| Microcrystalline | 8.25 |
| Cellulose | |
| Sodium Starch | 2.40 |
| Glycolate | |
| Magnesium Stearate | 0.72 |
| Lactose (Fast Flo ™) | 47.75 |
| Total | 96.46 |

The double layer tablet weight is: 446.46 mg.

Dry Blend Preparation Prior to Compression:

After blending the dispensed amount of tramadol HCl, xanthan gum and lactose for 20 minutes, the dispensed and sieved amount of magnesium stearate and silicium dioxide were added and blended for further 3 minutes. According to the topiramate strength needed in tablets the topiramate granules (with a drug load of 31.1% Topiramate) were diluted by adding lactose to the desired strength. The tramadol HCl dry blend and the topiramate blend were compressed to double layer tablets on a double layer rotary press tabletting machine.

### Example 2

Preparation of Tramadol HCl Pellets.

A dry blend of 1400 mg of tramadol hydrochloride, 1400 mg of microcristalline cellulose and 1200 mg of glyceryl benehate (Compritol 888 ATO™, Gattefosse) is wet massed with approximately 60 mg of water and extruded through a small orifice (approx. 1 mm). The extruded material is placed into a spheroniser where it is spun at high speed (pellet speed of between 5 and 20 ms⁻¹). During this step the extrudate breaks and rounds into pellets, the size being determined by the size of the extrusion orifice. The pellets are coated uniformly with the coating material sold under the trade name Surelease™ (Colorcon), which is a dispersion of ethylcellulose.

Preparation of Topiramate Pellets

A blend of 1400 mg of topiramate, 1400 mg of microcristalline cellulose and 1260 ml is wet massed and extruded through a small orifice (approx. 1 mm). The extruded material is placed into a spheroniser where it is spun at high speed (pellet speed of between 5 and 20 ms⁻¹). During this step the extrudate breaks and rounds into pellets, the size being determined by the size of the extrusion orifice. The pellets are coated uniformly with the coating material sold under the trade name Surelease™ (Colorcon).

3000 mg of the tramadol HCl pellets and 1000 mg of the topiramate pellets, both as prepared above are mixed. The thus prepared spherical pellet mixture is filled into capsules, at 128.6 mg of the mixture/capsule, using standard filling equipment.

1. A controlled release oral pharmaceutical preparation suitable for dosing every 24 hours comprising a substrate comprising a pharmaceutically effective amount of tramadol or a salt thereof and a pharmaceutically effective amount of topiramate; said preparation having a dissolution rate in vitro when measured using the Ph. Eur. Paddle Method at 75 rpm in 900 ml 0.05 M phosphate buffer at 37° C. and using HPLC:
between 15 and 35% tramadol released after 1 hour;
between 28 and 48% tramadol released after 2 hours;
between 47 and 67% tramadol released after 4 hours;
between 68 and 88% tramadol released after 8 hours;
between 79 and 99% tramadol released after 12 hours;
between 86 and 105% tramadol released after 18 hours;
about 100% tramadol released after 24 hours; by weight,
said preparation providing a therapeutic effect for about 24 hours after oral administration.

2. An oral pharmaceutical preparation as in claim 1, said preparation having a dissolution rate as follows:
between 20 and 30% tramadol released after 1 hour;
between 33 and 43% tramadol released after 2 hours;
between 52 and 62% tramadol released after 4 hours;
between 73 and 83% tramadol released after 8 hours;
between 84 and 94% tramadol released after 12 hours;
between 91 and 100% tramadol released after 18 hours;
about 100% tramadol released after 24 hours; by weight.

3. An oral pharmaceutical preparation as in claim 1, said preparation having a dissolution rate as follows:
25% tramadol released after 1 hour;
38% tramadol released after 2 hours;
57% tramadol released after 4 hours;
78% tramadol released after 8 hours;
89% tramadol released after 12 hours;
95,5% tramadol released after 18 hours;
about 100% tramadol released after 24 hours; by weight.

4. An oral pharmaceutical preparation according to claims 1 wherein the tramadol salt is tramadol hydrochloride.

5. An oral pharmaceutical preparation according to claim 1, wherein said substrate is a suitable matrix material in which tramadol or its salt form is incorporated.

6. An oral pharmaceutical preparation according to claim 5 wherein thematrix material is xanthan gum.

7. An oral pharmaceutical preparation according to claim 1, wherein the preparation comprises two or more phases.

8. An oral pharmaceutical preparation according to claim 7 wherein the major part of the tramadol or its salt form and of the topiramate are in different phases of the said pharmaceutical preparation.

9. An oral pharmaceutical preparation according to claim 7 wherein the tramadol or its salt form and the topiramate are in different phases of the said pharmaceutical preparation.

10. An oral pharmaceutical preparation according to claim 9 wherein at least one phase contains the major part of tramadol or a salt thereof and at least another phase contains the major part of topiramate.

11. An oral pharmaceutical preparation according to claims 7 wherein the phases take the form of layers and at least one layer contains tramadol or a salt thereof and at least another layer contains topiramate.

12. An oral pharmaceutical preparation according to claims 1, which has two layers, one layer containing topiramate and another layer containing tramadol or a salt thereof.

13. An oral pharmaceutical preparation according to claims 1 which has three layers, one layer containing topiramate, another layer containing tramadol or a salt thereof, and a third isolator layer in between the tramadol and the topiramate layers.

14. An oral pharmaceutical preparation according to claims 7 wherein at least one of the phases take the form of pellets.

15. An oral pharmaceutical preparation according to claim 14 wherein all phases take the form of pellets.

16. An oral pharmaceutical preparation according to claims 14 wherein the pellets contain either tramadol or a salt thereof or topiramate.

17. (canceled)
